(19) United States
(12) Patent Application Publication
(10) Pub. No.: US 2024/0136074 A1
(43) Pub. Date: Apr. 25, 2024

(54) TOOL FOR SUPPORTING THE INFORMED CONSENT OF PATIENTS AHEAD OF A MEDICAL TREATMENT

(71) Applicant: Bayer Vital GmbH, Leverkusen (DE)

(72) Inventor: Jens LIPINSKI, Rösrath (DE)

(21) Appl. No.: 18/277,520

(22) PCT Filed: Oct. 17, 2021
(86) PCT No.: PCT/EP2021/078818

§ 371 Date: Aug. 15, 2023

(30) Foreign Application Priority Data
Feb. 17, 2021 (EP) 21157561.8

(51) Int. Cl. G16H 80/00; G16H 10/20; G16H 10/60; G16H 40/60

(52) U.S. Cl. CPC G16H 80/00; G16H 10/20; G16H 10/60; G16H 40/60

(57) ABSTRACT

The present invention deals with the technical field of providing information to patients, for example prior to the performance of a medical treatment, and the electronic documentation of the provision of information. The subjects of the present invention are a computer system and a computer program product that make it possible to issue information to a patient in the form of selected information units and to detect and to document the selection and the behaviour of the patient when the information units are issued in order to issue them to a doctor at a later time so that said doctor is able to use this information for a needs-oriented discussion with the patient ahead of the medical treatment.

### CROSS REFERENCE TO RELATED APPLICATIONS

This application is a national stage application under 35 U.S.C. § 371 of International Application No. PCT/EP2021/078818, filed internationally on Oct. 18, 2021, which claims priority to and benefit of EP Patent Application No. 21157561.8, filed Feb. 17, 2021.

### FIELD

The present invention relates to the technical area of providing information to patients, for example before carrying out a medical treatment, and documenting the conveyance of information electronically.

### BACKGROUND

The principle of autonomy, which is enshrined in fundamental rights, guarantees every person freedom of decision, particularly with regard to medical treatment. To be able to decide autonomously, the person has to be made capable of making a reasonable decision for himself. Informed consent, also called informed agreement, informed assent, or consent after information has been provided, designates in the context of a medical treatment the consent of the patient, supported by information and explanation, to interventions and other medical measures.

Informed consent includes, in addition to the actual consent to a medical treatment, providing information and taking into consideration the desires, goals, and values of the patient in the decision process. The German Law on patient's rights explicitly requires, for example, providing information comprehensible to the patient (see German parliament (2013), Law on improving the rights of patients. German Federal Law Gazette (9): 277-82). In that respect, the requirements for comprehension are fundamentally to be understood as recipient oriented. Accordingly, “comprehensible” means that the provided information has to be linguistically comprehensible to the patient (http://dip21.bundestag.de/dip21/btd/17/104/1710488.pdf). This means that the information has to be provided in a language which physician and patient speak and understand adequately. In addition, the provided information has to be comprehensible in content and thus cannot contain excess jargon of the attending physician. Finally, information has to be provided in simple language and must repeated if necessary if the patient has difficulty comprehending the content of the information provided due to his physical, mental, or spiritual state.

The attending physician has to explain “all circumstances essential for the consent” (see German parliament (2013), Law on improving the rights of patients. German Federal Law Gazette (9): 277-82). These circumstances “generally include in particular the type, extent, performance, expected consequences and risks of the measures, and their necessity, urgency, suitability, and prospects of success with regard to diagnosis or treatment.” When providing information, “alternatives to the measure are also to be indicated if several methods which are similarly medically indicated and typical can result in significantly different stresses, risks, or prospects of recovery.” The patient has to be capable of deciding freely whether he will subject himself to the intervention. Obtaining informed consent is both an ethical and a legal obligation of physicians.

Providing information to the patient thus comprises the themes of diagnosis information, progress information, risk information, and information about the treatment alternatives. The largest part of providing information is typically occupied by risk information. A physician does not have to provide information about every risk; rather, the physician may convey to the patient information about those which can severely burden him in the course of his life and which the physician cannot assume that the patient expects.

The demands on the physicians increased because the case-law in Germany has not set a fixed numeric ratio between information obligation and complication rate, but stipulated the content and scope on the basis of criteria such as urgency and necessity of the intervention and probability of the occurrence of risks and consequences of risks on the course of life of the patient, but also due to pressure from the supervisory authorities, hospital administrations, and liability insurers.

The concept of the stepwise provision of information was developed in the 70s in Germany as a pragmatic compromise between a total provision of information, which will probably confuse the patient, and a strict individual provision of information, which is possibly problematic for the physician. For this purpose, a brochure or an information sheet having the most important information on the course and the risks of the planned intervention is given to the patient before the actual information conversation (first step). The information conversation itself (second step) is then used to answer questions which have arisen for the patient upon reading through the preliminary information, to explain individual circumstances, and for the patient to declare that he has no further questions and consents to the intervention.

By virtue of the fact that, in the information conversation, “reference can additionally also be made to documents which the patient receives in text form”, this concept of providing information is also legitimized in Germany in the new patient's rights law (see German parliament (2013), Law on improving the rights of patients. German Federal Law Gazette (9): 277-82).

### SUMMARY

The following are regarded as points of criticism of the stepwise provision of information:
a significant part of the provision of information is anticipated by handing out the written information,
the provision of information is formalized to such an extent that it is no longer possible to address the individual situation, and
information is provided beyond what is necessary in the specific case, the tendency toward total provision of information is strengthened, and the written information handed out therefore unnecessarily burdens the patient (see, for example, W. Wachsmuth, H. L. Schreiber: Die Stufenaufklärung—ein ärztlich und rechtlich verfehltes Modell [Stepwise provision of information—a medically and legally failed model], Chirurg 53(9) (1982): 594-8).

It is also questioned whether it is ensured by the signature of the patient that the patient has correctly understood the written information and has no further questions (see, for example W. Wachsmuth, H. L. Schreiber: Die Stufenäufklarung—ein ärztlich und rechtlich verfehltes Modell [Stepwise provision of information—a medically and legally failed model], Chirurg 53(9) (1982): 594-8).

Informed consent often has an unsatisfactory course in practice, both for the physician and the patient. Some patients understand the consent declaration solely as a legal requirement on the part of the physician and a necessary condition for carrying out a medical treatment (see, for example, D. E. Hall: Informed consent for clinical treatment, CMAJ. 2012 Mar. 20; 184(5):533-40. doi: 10.1503/cmaj.112120. Epub 2012 Mar. 5. PMID: 22392947; PMCID: PMC3307558),

A signed declaration does not necessarily indicate consent here: the patient who has signed a consent without completely understanding the course, utility, and risks of a treatment has not given informed consent (see, for example: A. Sherlock A et al.: Patients' recollection and understanding of informed consent: a literature review, ANZ J Surg. 2014 April; 84(4):207-10. doi: 10.1111/ans.12555. PMID: 24812707).

It has been shown again and again in practice that neither physicians nor patients are sufficiently prepared for all elements of the consent process (see, for example: M. Brezis et al.: Quality of informed consent for invasive procedures, Int J Qual Health Care. 2008 Oct.;20(5):352-7. doi: 10.1093/intqhc/mzn025. Epub 2008 Jul. 14. PMID: 18625699).

There is no real consensus about what form good provision of information can or should take in daily practice, and the fear of legal consequences has had the result that consent forms have been developed which serve less to provide information to the patient than to protect the physician (see, for example: M. I. Choudry et al.: Documenting the process of patient decision making: a review of the development of the law on consent, Future Hosp J. 2016 June; 3(2):109-113. doi: 10.7861/futurehosp.3-2-109. PMID: 31098199; PMCID: PMC6465837).

Typical consent forms are often unsuitable, because the information is offered in complex and poorly comprehensible language (T. M. Grundner: On the readability of surgical consent forms, N Engl J Med. 1980 Apr. 17; 302(16):900-2. doi: 10.1056/NEJM198004173021606. PMID: 7360176).

The comprehension of the patient is dependent on various factors such as age, education, intelligence, cognitive capabilities, locus of control, and anxiety (see, for example: M. M. Hutson, J. D. Blaha: Patients' recall of preoperative instruction for informed consent for an operation, J Bone Jt Surg Am 73(2) (1991): 160-2; M. Lashley et al.: Informed proxy consent: Communication between pediatric surgeons and surrogates about surgery, Pediatrics 105(3 Pt 1) (2000): 591-7); L. W. Morgan, I. R. Schwab: Informed consent in senile cataract extraction, Arch Ophthalmol 104(1) (1986): 42-5).

Over 50% of Germans are overwhelmed by health-relevant information; the rate is 66.3% for those over 65 years old, and is even 70.5% for immigrants (see, for example: R. Woratschka: Jeder Zweite versteht seinen Arzt nicht [Every other person does not understand their physician], http://www.tagesspiegel.de/politik/studie-zur-gesundheitskompetenz-jeder-zweite-versteht-seinen-arzt-nicht/13593996.html (last retrieved on 21.11.2016)).

Patients differ in their need and their processing of information (see, for example: M. T. Bowden et al.: Informed consent in functional endoscopic sinus surgery. The patient's perspective, Otolaryngol Head Neck Surg 131(1) (2004): 126-32).

Patients often do not feel the need more information and their knowledge about the utility and risks of a treatment remains low (see, for example: A. T. Ghulam et al.: Patients' satisfaction with the preoperative informed consent procedure: A multicenter questionnaire survey in Switzerland, Mayo Clin Proc 81(3) (2006): 307-12).

Patients are often not interested at all in the forms and do not read them carefully, if at all (see, for example: C. Lavelle-Jones et al.: Factors affecting quality of informed consent, BMJ. 1993 Apr. 3; 306(6882): 885-890, doi: 10.1136/bmj.306.6882.885).

The present invention is directed to these and further problems.

Provided is a computer system for conveying medical information and for acquiring the type and the scope of the conveyed medical information in the context of an informed consent of a patient to a medical treatment. In some embodiments, the computer system is configured
to receive inputs from the patient, wherein the inputs specify,
   which information units are to be output to the patient,
   in which sequence the information units are to be output, and
   in which form the information units are to be output,
to output the information units according to the inputs of the patient, wherein each information unit is designed such that it comprises written and/or spoken text, wherein the total number of the characters of the text in written form is less than 500,
to acquire the following information:
   which information units were not or were not completely output to the patient and/or which information units the patient has skipped,
   whether and possibly which information units were not output as a result of an input of the patient in spite of a notification of their importance,
   whether and possibly which questions and/or comments and/or assessments the patient has given on one or more information units, and
   how much time the patient has taken with consuming individual information units,
to document the received inputs and acquired information, and
to output the documented inputs and information to a physician.

Additionally, provided is a computer program product for conveying medical information and for acquiring the type and the scope of the conveyed medical information in the context of an informed consent of a patient to a medical treatment. In some embodiments, the computer program product comprises a computer-readable data carrier and program code, which is stored on the data carrier and which, upon execution on a computer system, causes the computer system to carry out the following steps:
receiving inputs from the patient into the computer system, wherein the inputs specify:
   which information units are to be output to the patient,
   in which sequence the information units are to be output, and
   in which form the information units are to be output,
outputting the information units according to the inputs of the patient, wherein each information unit is designed such that it comprises written and/or spoken text, wherein the total number of the characters of the text in written form is less than 500,
acquiring the following information:
   which information units were not or were not completely output to the patient and/or which information units the patient has skipped,
   whether and possibly which information units were not output as a result of an input of the patient in spite of a notification of their importance,
   whether and possibly which questions and/or comments and/or assessments the patient has given on one or more information units, and
   how much time the patient has taken with consuming individual information units,
documenting the received inputs and acquired information, and
outputting the documented inputs and information to a physician.

A computer-implemented method for conveying medical information and for acquiring the type in the scope of the conveyed medical information in the context of an informed consent of a patient to a medical treatment is also described. In some embodiments, the method comprises the following steps:
receiving inputs from the patient, wherein the inputs specify:
   which information units are to be output to the patient,
   in which sequence the information units are to be output, and
   in which form the information units are to be output,
outputting the information units according to the inputs of the patient, wherein each information unit is designed such that it comprises written and/or spoken text, wherein the total number of the characters of the text in written form is less than 500,
acquiring the following information:
   which information units were not or were not completely output to the patient and/or which the patient has skipped,
   whether and possibly which information units were not output as a result of an input of the patient in spite of a notification of their importance,
   whether and possibly which questions and/or comments and/or assessments the patient has given on one or more information units, and
   how much time the patient has taken with consuming individual information units,
documenting the received inputs and acquired information, and
outputting the documented inputs and information to a physician.

Preferred embodiments of the present invention are found in the dependent claims, in the present description, and in the drawings.

### BRIEF DESCRIPTION OF THE FIGURES

The invention will now be described, by way of example only, with reference to the accompanying drawings.

FIG. 1 schematically shows an example embodiment of the computer system according to the invention.

FIG. 2 shows a possible breakdown of themes (levels of detail) for the treatment of an age-related macular degeneration (AMD) by intra-vitreal surgical administration of medication (IVOM).

FIGS. 3(a)-3(f) show examples of display screen contents of the computer system, according to some embodiments.

FIGS. 4(a)-4(h) show examples of buttons that may be provided in a display screen of the computer system, according to some embodiments.

FIGS. 5(a)-5(c) show examples of various information types from which the user can select, according to some embodiments.

FIGS. 6(a)-6(c) show example displays on a computer system according that is embodied as a smart phone or tablet computer, according to some embodiments.

FIG. 7 shows an example display on a computer system that is embodied as a smart phone or tablet computer. according to some embodiments.

FIG. 8 shows example information that has been acquired and documented during the conveyance of information by the computer system and the computer program according to the invention, according to some embodiments.

FIG. 9(a)-9(b) show example displays on a display screen of a computer system that embodied as a smart phone or tablet computer, according to some embodiments.

FIG. 10 shows a device operated by a patient, according to some embodiments.

### DETAILED DESCRIPTION

The invention is explained in more detail hereinafter, without distinguishing between the subjects of the invention (computer system, computer program product, computer-implemented method). Rather, the following explanations are to apply for all subjects of the invention analogously, independently of the context in which they are made (computer system, computer program product, computer-implemented method).

The invention is explained in more detail at some points in the description with reference to drawings and/or examples, without the intention to restrict the invention to the features and combinations of features shown in the drawings and/or mentioned in the examples.

Furthermore, the various features mentioned and shown in the description and the drawings can be combined with one another and the present invention is also intended to include each of these combinations.

The present invention provides means for conveying medical information to a patient according to the specifications of the patient and permits a physician to give detailed information about which information the patient has actually consumed, in which form the patient consumed said information, and which questions he has on individual information and/or in relation to which individual information the patient could have a further requirement for conversation. The present invention therefore represents, on the one hand, an instrument for conveying information; on the other hand, however, it can also be understood as a sensor which analyzes the behavior of the patient in the context of conveying information to a physician about which information the patient has (particular) interest in, which information the patient has little or no interest in, and which information the patient has possibly not understood or not understood completely. The present invention can be understood as a sensor which gives the physician information about whether the patient can give an informed consent or whether and possibly in which areas further provision of information could be necessary/reasonable and possibly in which manner assisted by speech or graphics the physician can give this to the patient. The present invention therefore does not provide means for solely (passively) conveying information according to the specifications of a patient, rather it (actively) analyzes the decisions and the behavior of the patient during the conveyance of information and provides the information obtained during the analysis to a further person (typically a physician).

The conveyance of information according to the invention takes place in the context of the informed consent of a patient to an upcoming medical treatment. The patient has to decide whether a medical treatment will be performed on him or with him.

The medical treatment can be a therapy, a diagnostic measure, an operation, a prophylactic measure, or another medical treatment, including participating in a clinical study.

The present invention provides that the information is conveyed by means of a computer program, often also colloquially referred to as an app (short form of application). The computer program (also called program code) is stored on a data carrier (for example on a hard drive or a USB stick or a CD-ROM). The program code of the computer program product according to the invention causes the computer system according to the invention to carry out the steps described in this description when the program code is loaded in a working memory of the computer system and executed.

A “computer system” is an electronic data processing system that processes data by means of programmable computing rules. Such a system typically comprises a “computer”, the unit which comprises a processor for carrying out logical operations, and peripherals. “Peripherals” in computer technology designate all devices which are connected to the computer and which are used to control the computer and/or as input and output devices. Examples thereof are monitor (screen), printer, scanner, mouse, keyboard, drives, camera, microphone, speakers, etc. Internal ports and expansion cards are also regarded as peripherals in computer technology.

Modern computer systems are frequently divided into desktop PCs, portable PCs, laptops, notebooks, netbooks, and tablet PCs, and what are called handhelds (e.g. smartphones); all these devices can be utilized for execution of the invention.

The computer system of the present invention can comprise one computer or multiple computers (which are separate, i.e., spatially separated from one another).

Inputs in the form of control commands and/or data into the computer take place via input means such as a keyboard, a mouse, a microphone, a touch-sensitive display screen, a touchpad, but also by transferring from another computer via a network connection and/or reading from an internal or external data memory and/or the like. “Input” is also to be understood as meaning the selection of an entry from a virtual menu or from a virtual list or clicking on a checkbox and the like by the user of the computer system according to the invention.

Outputs typically take place via a display screen (monitor), on a printer, via loudspeaker (including headphones), and/or by storage on an (internal and/or external) data memory. Data which are collected and/or acquired by means of the computer system/computer program according to the invention are preferably stored in a cloud memory.

The term “cloud computing” is understood as the availability of computer system resources on demand, in particular data storage and computing performance, without direct active management by the user. The term is generally used to describe computing centers which are available to many users via the Internet. In other words: Cloud computing is the provision of computer services—including servers, data memories, databases, networks, software, analyses, and information—via the Internet (“the cloud”), to offer flexible resources and have data and services available at various locations.

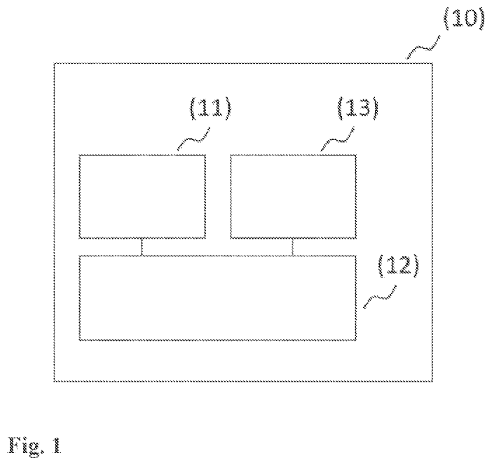

FIG. 1 schematically shows by way of example an embodiment of the computer system according to the invention. The computer system (10) comprises an input unit (11), a control and calculation unit (12), and an output unit (13). A user can use the input unit (11) to input information and control commands into the device. Information can be output to a user via the output unit (13), preferably displayed on a monitor and/or output via a loudspeaker. The control and calculation unit (12) is used primarily to control the components of the device (10), to process the information that has been input and output and to perform calculations and logical operations.

The computer system according to the invention is configured (by means of the computer program according to the invention) to give a user (the patient) information about an upcoming medical treatment.

The information can be displayed on a display screen of the computer system according to the invention in the form of text, graphics, and/or images and/or output via a loudspeaker (including headphones), for example as a speech communication. A combination of displayed text and spoken speech is also possible. The display of video sequences on a display screen, optionally in combination with the output of spoken information via a loudspeaker (including headphones), is also conceivable. Outputs on a braille output device are also conceivable.

In this description, the term “output of information” is used for the computer-assisted conveyance of information, which is to include all abovementioned types of conveyance of information and further ones.

The computer system according to the invention is configured to output medical information in the form of information units to a user, to receive inputs from the user, and to acquire information on the behavior of the user during the conveyance of information.

The information is output in the form of information units. Each information unit comprises written and/or spoken (read aloud) text. Independently of whether the text is displayed in written form on a display screen of the computer system according to the invention or is output by speech output via a loudspeaker of the computer system according to the invention, the text preferably has fewer than 500 characters (including spaces). The maximum number of the characters is preferably below 400 (including spaces). The average (arithmetic mean) number of the characters per information unit, averaged over all information units, which a user can consume, is preferably below 300 characters (including spaces).

In one embodiment, an information unit preferably comprises not more than four, preferably not more than three, particularly preferably not more than two feature contents (key messages). A feature content is typically an amount of information which may answer a question as to what the respective feature content is with at most three main sentences, preferably with only one main sentence. An information unit preferably comprises not more than 5 sentences. An information unit preferably comprises not more than 60 words, preferably not more than 50 words. The information units are preferably designed so that the total content of an information unit in text form having a capital letter height of 1.8 mm to 2.5 mm on a display screen of an Apple iPhone 6s can be displayed on the display screen such that the user does not have to scroll the text to be able to read the entire text.

The information units preferably have a scope (a length), which permits the user to consume (i.e. read and/or view and/or hear) each information unit in a defined time span. This time span is at most 120 seconds, preferably at most 90 seconds, still more preferably at most 60 seconds.

If the information of an information unit is output via a speech output (via loudspeaker) and/or in the form of an animation and/or video sequence to the user, the output then has a defined length, which is preferably in the range of 10 seconds to at most 120 seconds, still more preferably in the range of 10 seconds to at most 90 seconds, still more preferably in the range of 10 seconds to at most 60 seconds per information unit. If the computer system is configured so that the user can increase and/or decrease the speed of the speech output and/or the animation and/or the video sequence (to be able to adapt it to his personal requirements), the time span for the output of the information is then for this case at most 120 seconds, still more preferably at most 90 seconds.

The average output duration (arithmetic mean) of an information unit, averaged over all information units which a user can consume, is preferably in the range of 20 seconds to 60 seconds.

The average reading speed of written information for users is typically higher than the reading-aloud speed for a professional speaker, since the reading user only has to acquire the text with the eyes and does not have to say it aloud. If the information is only output as text to be read, the amount of text per information unit is then restricted such that a user can read through the text in at most 60 seconds, preferably in at most 50 seconds, still more preferably in at most 40 seconds, most preferably in at most 30 seconds.

In ascertaining the average reading speed of users, an average value (arithmetic mean) of a large number of readers can be determined.

The average reading duration (arithmetic mean) of an information unit, averaged over all information units, is preferably in the range of 15 seconds to 50 seconds.

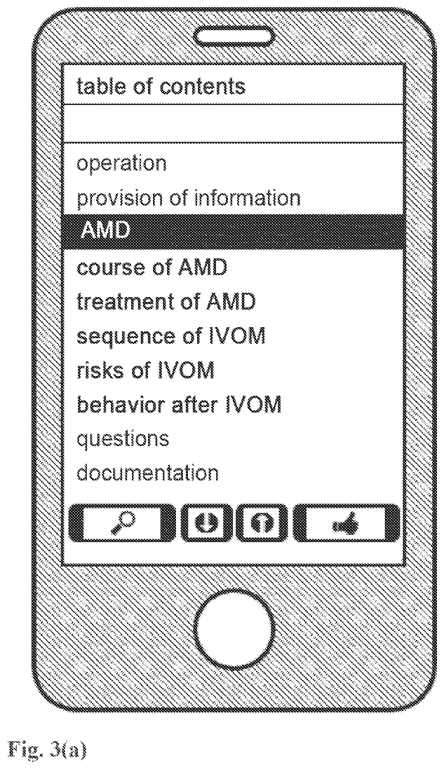

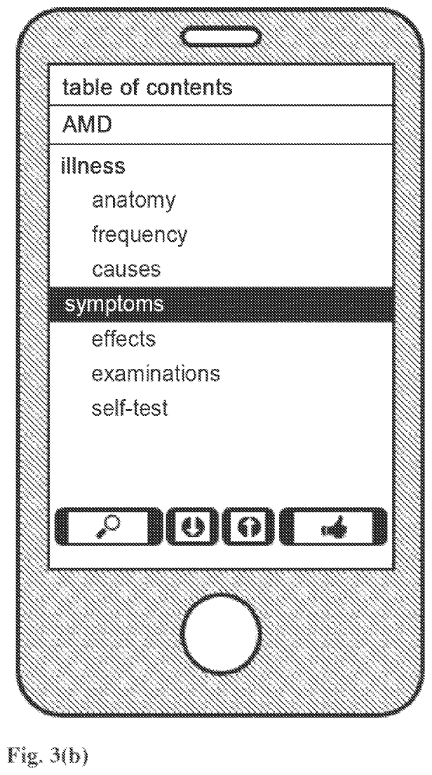

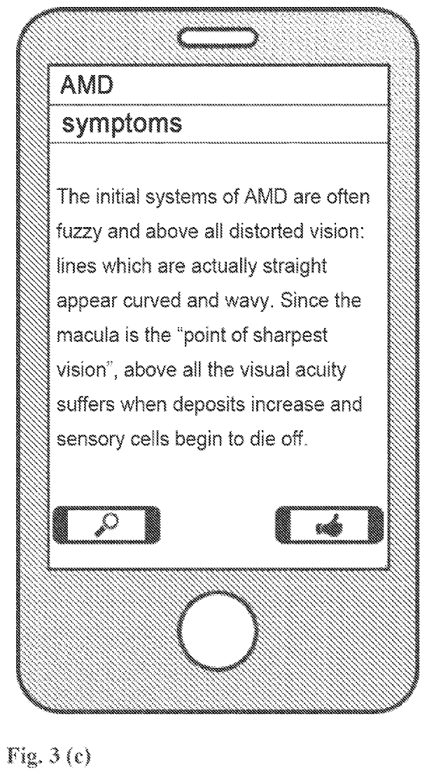

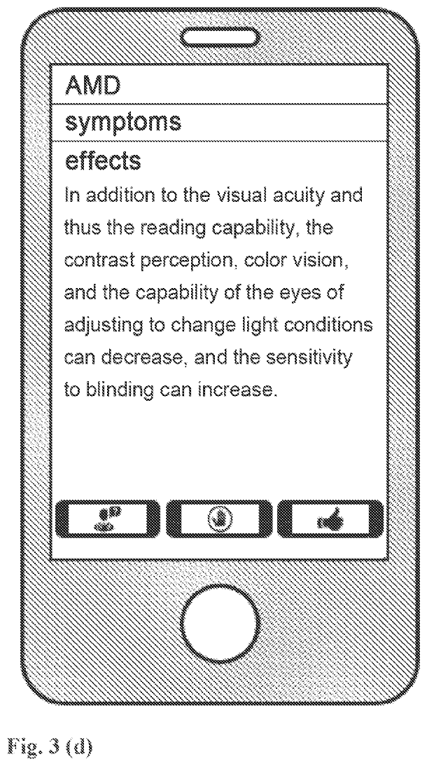

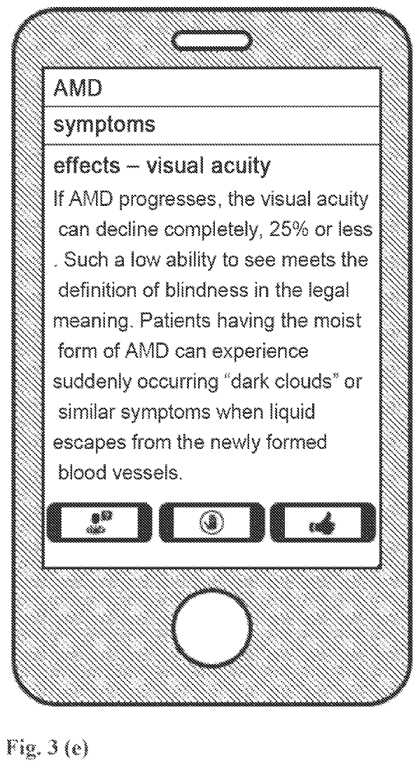

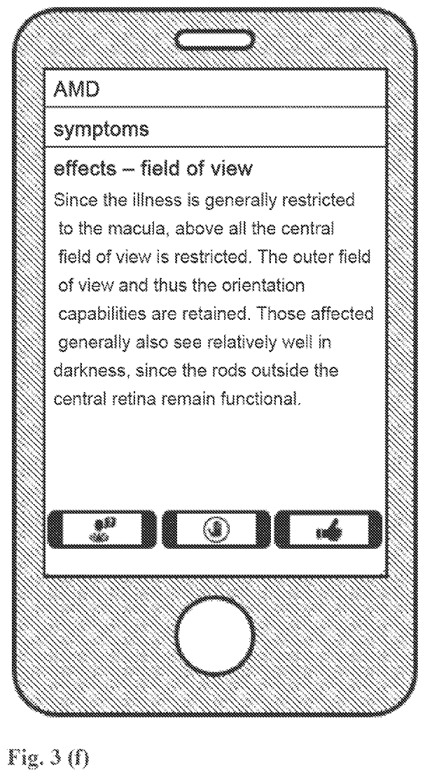

The information units shown in FIGS. 3(c), 3(d), 3(e), and 3(f) are examples of such “short information units” according to the invention.

These comparatively short information units (in comparison to a medical information provision brochure) have various advantages: their scope (their length) does not exceed the typical concentration capability of a user. The user can consume individual information units and decide himself whether he wishes and/or can consume one or more further information units or whether he wishes to and/or has to take a break. In addition, the comparatively short information units permit more accurate acquisition of what the user has consumed. The comparatively short information units thus not only have the effect that the user can simply digest the information units, rather that it is also recognizable which information has been output to the user so that, for example, a physician can use this for a subsequent conversation with a patient (the user).

Furthermore, dividing the information into small “heaps” (information units) facilitates a more detailed insight into the use of the computer program/computer system according to the invention by the user. More detailed information can be collected about which information units the user has consumed, which ones he has skipped, how long he has required for consuming the individual information units, which questions/comments he has noted with respect to individual information units, which information units the user has classified (for example marked) as useful or less useful or incomprehensible, in which sequence the user has consumed information units, and/or the like. All of these and possibly further items of information give the physician insight into the extent to which a patient has understood a planned medical treatment, is prepared for it, and can give informed consent to the performance of it. The information can also help the developer/operator of the computer program according to the invention to optimize the computer program according to the invention, however, in order to ultimately better address the needs of the user and be able to adapt the conveyance of information to the needs of the user.

The computer system according to the invention is preferably configured to acquire those information units which were output once and/or multiple times to the user, including the points in time at which they were output at all and/or for the last time.

The computer system according to the invention is preferably configured to acquire the sequence in which the information units were output. The acquisition of the sequence can be used to optimize the user guidance, for example, when it proves that information units are consumed by many users in a different sequence and/or with different content focal points than originally intended by the developer/operator. The sequence can also give the physician an indication, however, as to which prioritization the patient has performed in the conveyance of information, i.e., which information units the patient has particular interest in (and/or has less interest in) and which information units have caused him to retrieve (or to skip) specific other information units. If the user has had one information unit output multiple times, this can indicate that the user considers the content to be important and/or difficult to comprehend; questioning by the physician can give information here and the physician can then address the user's individual requirements in a targeted manner.

The computer system according to the invention is preferably configured to acquire those information units which were not and/or not yet output to the user. In the acquisition of the information units which have already been output, it can also be acquired how long the user has needed to consume the corresponding information units or how much time has passed between the retrieval of one information unit and the change to another information unit, since it is conceivable that the user has retrieved the information unit but not yet (completely) consumed it, rather (for whatever reason) has decided that he does not (completely) acquire the present information unit, rather wishes to change to another information unit. Retrieving an information unit does not necessarily mean that this information unit has actually been consumed.

The time for which an information unit has been displayed is preferably acquired. This time is compared to one reference time or multiple reference times. If there is a defined deviation of the display unit from the at least one reference time, it is noted (assumed) for the information unit that the user has consumed or has not consumed this information unit (with high probability). The reference time can be, for example, the time which a reader requires at least on average to completely read the information unit. If the acquired display time is less than the reference time (average reading time), it is probable that the user has not completely read the information unit. If the acquired display time is greater than the reference time (for example at least 50% higher), it is probable that the user has completely read the information unit. If the acquired display time is significantly greater than (for example more than 100%) the reference time (average reading time), it is conceivable that the user had problems in comprehending the information unit.

For information units which are so extensive (exceptionally or upon use of images and/or graphics) that they cannot be displayed completely on the display screen, and in which the display screen content on the display screen can be changed by “scrolling”, it can be acquired whether the entire content of the information unit has been displayed on the display screen by “scrolling” in order to decide whether an information unit has probably been consumed or has probably not been consumed.

For information which is output via a loudspeaker (including headphones), it can be acquired whether the entire audio content has been played back. This applies analogously to the playback of animations, video sequences, and/or image sequences. “Fast forwarding” of an output can also be acquired.

It is also conceivable that movements and/or locations of the computer system are required. It is conceivable, for example, that the user has the information units output via a tablet computer. This typically has an array of sensors, which give information about whether the device is moved and/or accelerated and how it is held (upright, crosswise, tilted forward (toward the user) or to the rear (away from the user), and the like). A computer system which is moved during the output of information units indicates that the user is active. An idle device can indicate that the user has laid the device to the side. Typical movement profiles may be created for users who engage (actively) with information units and those who consume information units more casually (passively). Typical user profiles may be created for users who can follow the information units and those who have difficulties with operating the computer program and/or with the output content. A comparison of the user behavior to the created profiles and the assignment of the user to one of the created profiles (for one or for multiple information units) can give the physician further information about the provision of information to the patient.

It is preferably output to the user which information units have already been output and which have not yet been output. This can take place in particular in the case of informed consent both during repeated processing of repeating medical measures and also during repeated processing of a single medical measure. It can also be displayed to the user hear how much time he has already used consuming the information units and how much he will require for those still remaining (expected value). The time span which the user has used for the information units already consumed is preferably taken into consideration in the calculation of the time span for the consumption of the remaining information units. For example, an average consumption speed of the user can be calculated. This can be compared to the average reading (aloud) duration or presentation speed. If it is longer, for example, by a factor x, the calculated remaining duration can be multiplied by this factor x. In this way, a value for the duration of the remaining information units is displayed to the user, which takes into consideration the individual speed of the user when consuming information units. On the one hand, a realistic value is thus displayed to the user, on the other hand, the acquisition of the average consumption speed helps in the documentation and the comprehension of the individual information processing and the needs of the user in the conveyance of information—the average consumption speed can thus also be important information for a physician.

The user can preferably mark individual information units, for example, if they appear important to him and/or he possibly wishes to consume them once again at a later point in time and/or if he has questions on information units which he wishes to clarify at a later point in time, for example with a physician (in case of providing information about a medical treatment). It is conceivable that the user can assess individual information units, for example, how useful the respective information unit was for him, how comprehensible it was, whether it has room for improvement with regard to comprehensibility, scope, possible redundancy, and/or depth of detail, etc. Such an assessment can be carried out, for example, by input of free text in a text field and/or by selection of prefinished assessment levels and/or the like.

The user can preferably make notes on individual information units in the form of text inputs and/or speech inputs. Such notes can comprise, for example, questions to the physician or comments which the user (patient) wishes to remember for specific tasks still to be accomplished before a medical treatment.

The user can preferably search for keywords via a search function, in order to deliberately arrive at an information unit which comprises and deals with the respective keyword.

The information units are preferably divided into themes. An overview in the form of a table of contents or the like about the themes is preferably given to the user. The overview is preferably designed so that the user can arrive directly at the respective theme by selecting a theme in the overview (for example, by clicking a header which represents the theme, by means of a finger on a touch-sensitive display screen, or by selection using a mouse or the input of a speech command). It is conceivable that individual or all themes are in turn divided into sections (themes).

Possible themes which can be dealt with in the computer-assisted provision of information to the patient according to the invention are one or more of the themes listed hereinafter:
Greeting and general information on the structure and operation of the application
General information on the goal, on content and sequence of a provision of information
Information on the cause, due to which a medical treatment is recommended, and on the untreated progression Information on the sequence of the medical treatment and the typical results of the medical treatment
Information on possible alternatives
Information on risks of the medical treatment and the risks of possible alternatives
Information on behavior before, during, and after the medical treatment, for example, on measures which the patient can/should take before/after the medical treatment and/or on the behavior of the patient before/after the medical treatment and/or changes which can happen to the patient and/or which the patient should be aware of, and because of which he should contact a physician if necessary
Documentation on the content of the provision of information and preparation of the physician conversation with possible consent declaration

In one preferred embodiment, information on the upcoming medical treatment and the indication for the upcoming medical treatment are offered to the patient in various levels of detail, so that the patient has the possibility in multiple levels one after another of “diving in” in each case into more details. Possible levels of detail are, for example, theme, section, chapter, paragraph, and keyword.

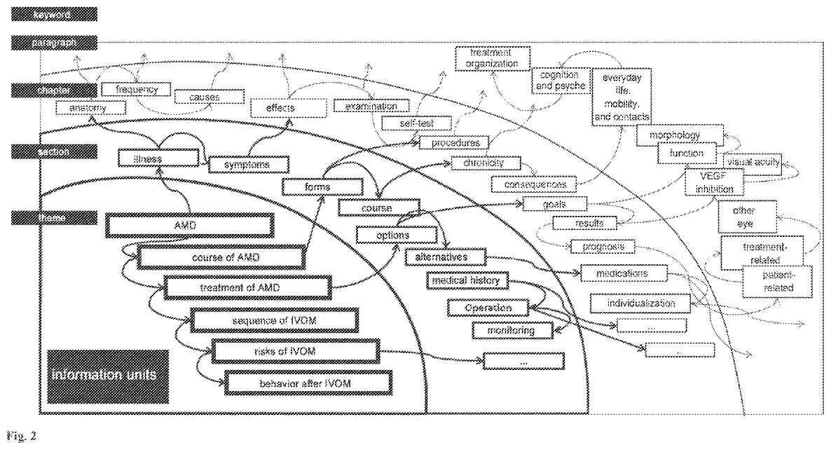

FIG. 2 shows a possible breakdown of themes (levels of detail) on the basis of an example: the treatment of an age-related macular degeneration (AMD) by intra-vitreal surgical administration of medication (IVOM).

The term ‘level of detail’ preferably means here that multiple information contents are offered in various degrees of detail. A respective higher level of detail is a summary of the lower levels of detail here. The user thus already experiences the most important on the upper level, experiences more on a lower level, and most on the lowest level.

FIGS. 3(a),(b),(c),(d),(e), and(f) show examples of display screen contents of the computer system according to the invention. The computer system according to the invention (or at least a part thereof) is embodied in these examples as a smart phone or tablet computer. FIG. 3(a) shows an overview of themes in the form of a table of contents. If a user selects the theme “AMD”, the display screen content of FIG. 3(b) is displayed to him. As shown in FIG. 3(b), the theme “AMD” is broken down into further themes and subthemes. If a user selects the further theme “symptoms”, the display screen content of FIG. 3(c) is displayed to him. FIG. 3(c) shows an information unit on the theme “AMD—symptoms”. FIG. 3(d), Figure (e), and Figure (f) each show a further information unit, which comprises more detailed information on the subtheme “AMD— symptoms—effects”.

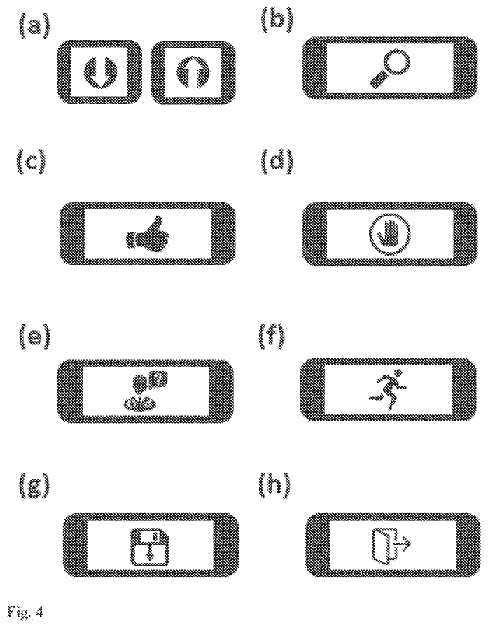

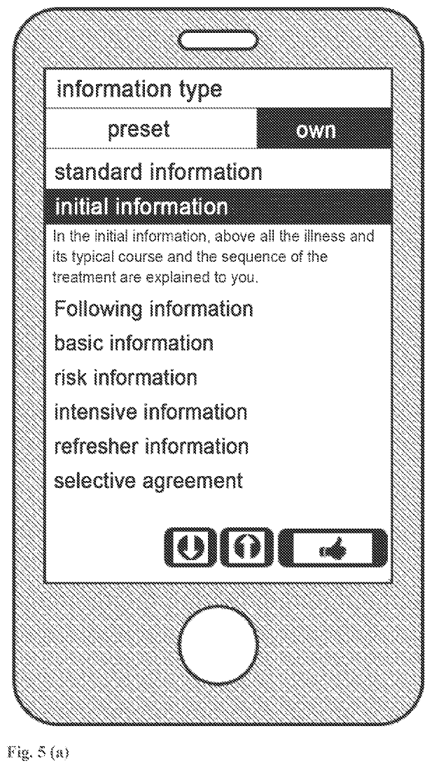

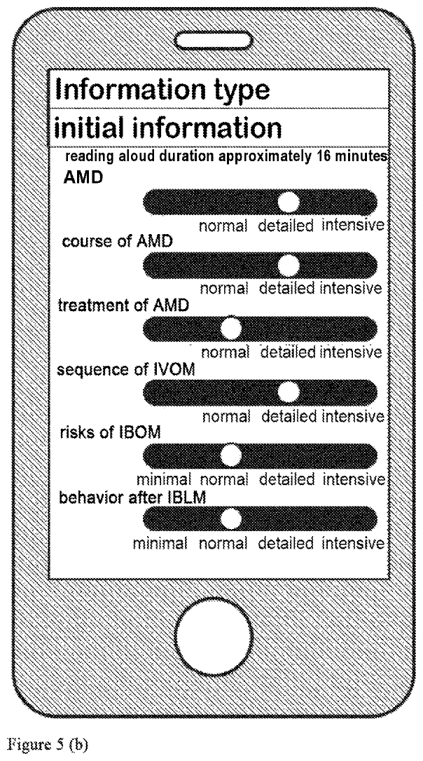

FIGS. 3(a),(b),(c),(d),(e), and(f) show a row of (virtual) buttons in the lower display screen area, via which the user can input commands into the computer system. These buttons are shown once again in FIG. 4(a),(b),(c),(d), and(e). Furthermore, further buttons are shown by way of example in FIG. 4 which the computer program according to the invention can include.

Using the arrow buttons in FIG. 4(a), a user can navigate from one selection field to the next (thus next higher or next lower) and/or shift the display screen content in the display upward or downward (scroll), to have information displayed which is associated with an information unit but is not completely displayed on the display screen due to the size of the information unit.

Using the button with the magnifying glass in FIG. 4(b), the user can retrieve more detailed information on the information unit which is presently displayed. A further information unit is displayed to him upon actuation of the virtual button, which provides further (more detailed) information on the presently handled theme.

Using the “okay” button in FIG. 4(c), the user can move to the next theme (in the sequence as indicated in the table of contents).

Using the “stop” button in FIG. 4(d), the user can skip the output information unit.

Using the “question” button in FIG. 4(e), the user can document a question, for example to a physician.

Using the “exit” button in FIG. 4(f), the user can move back to the theme overview and/or save the processing status.

Using the “save” button in FIG. 4(g), the user can save the processing status. All information acquired by the computer program according to the invention in the time between the call of the computer program according to the invention and the save or between the prior save and the current save is secured here

The user can close the application using the “exit” button in FIG. 4(g). An automatic save of the processing status preferably takes place here.

The inputs can also, as already described, be given via speech command by the user, for example in the form of the commands “up/down” (according to button of FIG. 4(a)), “details” (according to button of FIG. 4(b)), “next theme” (according to button of FIG. 4(c)), “skip” according to button of FIG. 4(d), “question” (according to button of FIG. 4(e)), “save” (according to button of FIG. 4(f)), and “close” (according to button of FIG. 4(h)). Furthermore, the information units can also be output to the user via speech output.

When conveying information in the context of an informed consent, the patient can preferably select whether he wishes to run through the provision of information about a medical treatment in a completely individualized manner, or according to various prepared (typical) information types. The user can preferably select one information type from a list of possible information types. The possible information types can differ here, for example, with respect to the scope of the output information and/or the selection of the information units to be output.

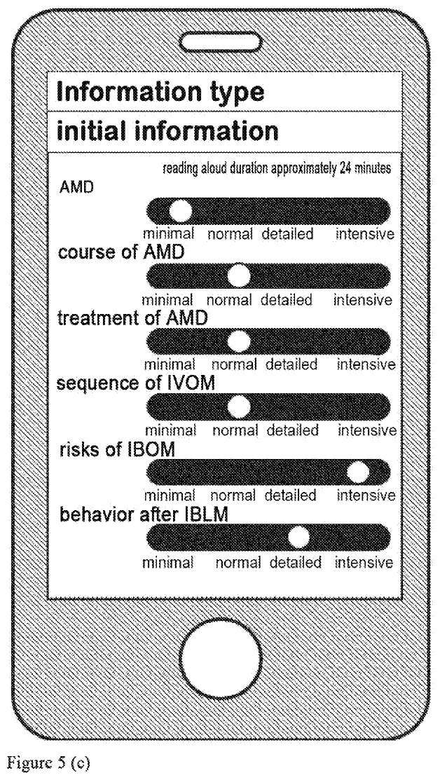

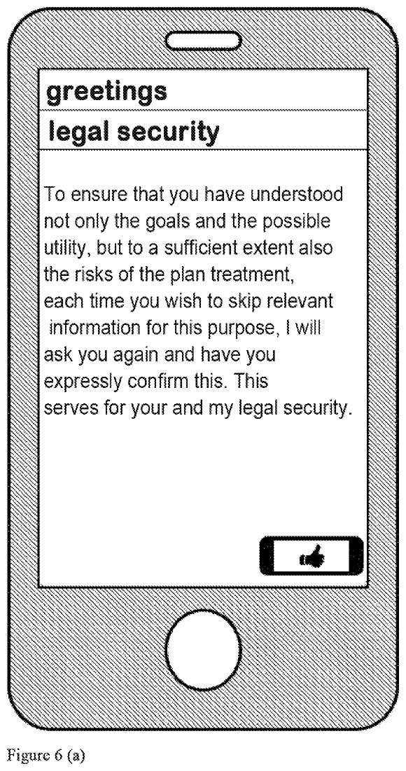

FIGS. 5(a),(b), and(c) show examples of various information types from which the user can select one. In FIG. 5(a), the various information types are specified by a header. The information types can be, for example: Standard information, initial information, follow-up information, basic information, risk information, intensive information, refresher information, and selective agreement. In a selective agreement, services outside the so-called regular care can be agreed upon. The individual physician enters into a contractual relationship here directly with the health insurance company. If the contracting physician and his patient are entered in the same selective agreement, the care no longer takes place via the regular care. The selective agreement is insofar a special information type, in that it typically not only has medical content, but can additionally include a form inserted by the physician, in which the patient affirms the possibilities of entering into indication-specific or treatment-specific contracts. This is typically the participation in care contracts specific to health insurance (which the physician also offers). Information and consents on practice-specific consents, for example data protection, can also be able to be integrated and retrieved in the information type.

It is conceivable that depending on the indication and/or the provided treatment and/or the patient type, fewer, more, and/or other information types are displayed and selectable. In the present example, a brief description about what characterizes the respective information type appears below the header for the respective information type. In the present example, the information type “initial information” is preselected and below the header “initial information”, a brief text having an explanation is displayed.

The information type “standard information” is typically the information type which is displayed to a patient when he does not make a selection of a specific information type. The standard information is typically the information type which the patient receives when he does not wish to navigate completely individually through the information, but rather wishes to have a recommended information flow (from the developer/operator/physician) offered. It is also important to note here that the patient can not only select and deselect focal points for each of the information types, but rather can deviate at any time from the proposed course by skipping proposed contents or retrieving more detailed information units upon interest.

FIG. 5(b) and FIG. 5(c) show by way of example on the basis of the information types “initial information” and “risk information” how the information types differ and by which features the information types can be characterized. Themes are shown in FIG. 5(b) and FIG. 5(c) which can be handled in the provision of information. A slide controller is located on the right of each theme. A scale is located below the slide controller, which is described by the attributes “minimal”, “normal”, “detailed”, and “intensive”. The reading aloud duration is displayed in the upper area of the display screen content; in this case this is the total reading aloud duration for consuming all information units. The reading aloud duration thus indicates what the respective selection of a degree of detail means for the total duration. The farther a slide controller for a specific theme is shifted to the right, the more extensively the theme will be handled and the longer the reading aloud duration generally is.

It is also conceivable that the user can define his own information type. A tab having the inscription “own” can be seen in FIG. 5(a) in the top right area of the displayed display screen. If the user selects this menu point, for example, he can freely determine the degree of detail of individual themes. By way of the above-described virtual slide controller, the user can determine himself, for example, how extensively a theme should be/will be handled.

As already described multiple times in this description, the inputs of the user also mentioned in the preceding sections are acquired by the computer system and can be displayed to a physician.

As already described, the computer system according to the invention can be configured so that the user (patient) can skip information units. The present invention therefore enables a patient to expressly dispense with elements of the provision of information in the scope of his right of self-determination (see German parliament (2013), Law on improving the rights of patients German Federal Law Gazette (9): 277-82).

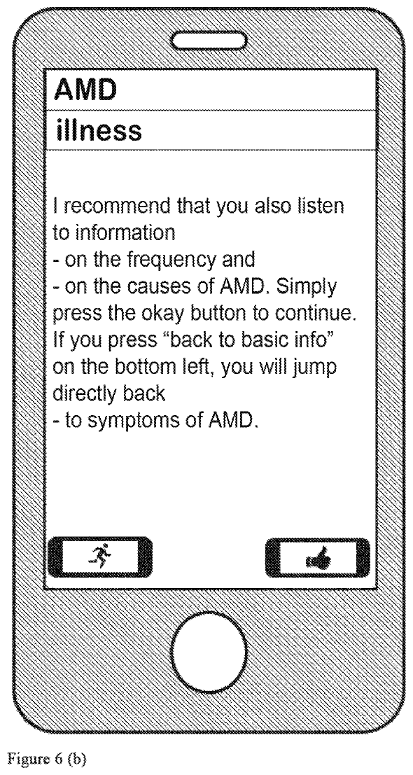

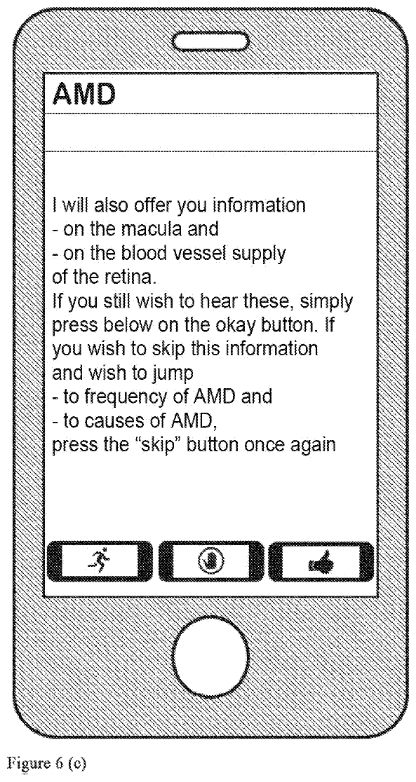

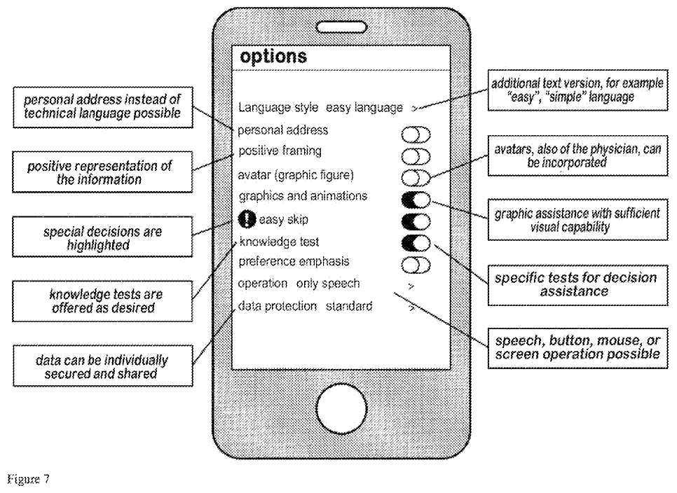

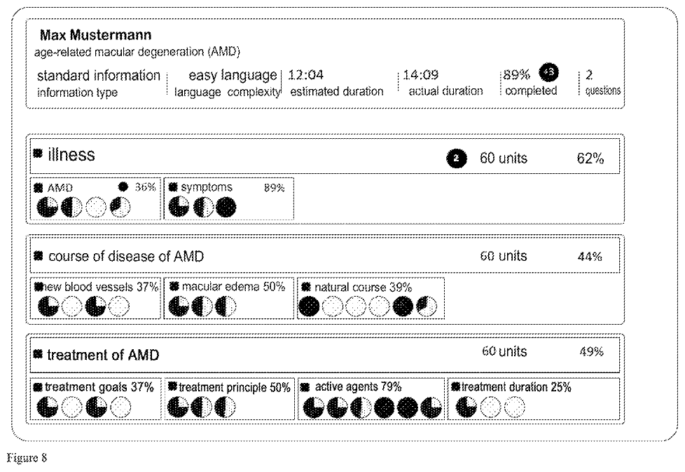

It is conceivable here that one or more information units which are particularly important from the viewpoint of the patient information cannot be skipped. It is furthermore conceivable that the user is made aware by a notification of the importance of one of more information units if he wishes to skip this one or these multiple information units, possibly also depending on the importance with notifications of different intensities. Furthermore, it is conceivable that recommendations for specific information units and/or offers for specific information units are given to the user. The functionalities described in this section are shown by way of example in FIGS. 6(a),(b), and(c). FIGS. 6(a),(b), and(c) show schematically and by way of example displays on a computer system according to the invention, which is embodied as a smart phone or tablet computer. Individual information units are displayed to a user on a display of the computer system.

In one preferred embodiment, the user can further influence the form of the information units/information conveyance.

The concept ‘form’ is understood in this description, for example, as the language, the language style used, the representation, the assistance of text by graphics, images, and/or animations, the output via a loudspeaker (including headphones) and/or a monitor, the form of address, and/or further forms and design options.

It is conceivable, for example, that the user can select the language style/the language complexity. A distinction can be made in the language style, for example, between technical language, standard language, simple language, and easy language.

Technical language is language applicable for a specific technical field or for a specific branch; it is generally not suitable for providing information to patients—but is typical in communication between physicians about medical content.

Standard language is understood as a generally binding form of speech which is spoken and written in public. It is also referred to as high-level language. It is typically used in school, at work, and in written communication. For more details, see, for example https://de.wikipedia.org/wiki/Standardsprache.

Texts in simple language have shorter sentences and simpler sentence structures than standard language and technical language. Foreign words, figures of speech which are difficult to understand, such as turns of phrase or metaphors, are not used in simple language, neither are unusual figurative phrases and plays on words. Texts in simple language enable a larger part of the population to access information or literature. It is important above all in simple language to adapt texts perceived as difficult to the reading competence of broader population groups. For more details, see, for example: https://de.wikipedia.org/wiki/Einfache_Sprache.

Easy language is a specially regulated simple language. The linguistic mode of expression is directed to particularly easy comprehensibility. A set of rules was issued by the German association Netzwerk Leichte Sprache [network for easy language]. In addition to language rules, it also comprises orthography rules and recommendations on typography. Easy language is intended to facilitate the understanding of texts for people who have a low level of competence in the language used (German, English, or the like) for various reasons. For more details see, for example: https://de.wikipedia.org/wiki/Leichte_Sprache.

It is also conceivable that the user can select a familiar form of address or a polite form address.

It is conceivable, for example, that the user can select whether the conveyance of information is to be neutral (in particular the medical provision of information) or more positive (positive framing).

It is conceivable, for example, that the user can select whether the conveyance of information is to be enriched by one or more knowledge tests, so that the user can recognize whether he has correctly understood all points of the conveyance of information.

It is conceivable, for example, that the user can select whether one or more avatars (for example an avatar of the physician) are to be incorporated in the conveyance of information to make the conveyance of information more personal.

It is conceivable, for example, that the user can select whether the provision of information is to be enriched by graphics and/or animations.

If information is to be conveyed via a speech output, the user can set the reading aloud speed, for example.

As already described multiple times in this description, the inputs/selections of the user mentioned in the preceding sections are also acquired by the computer system and can be displayed to a physician.

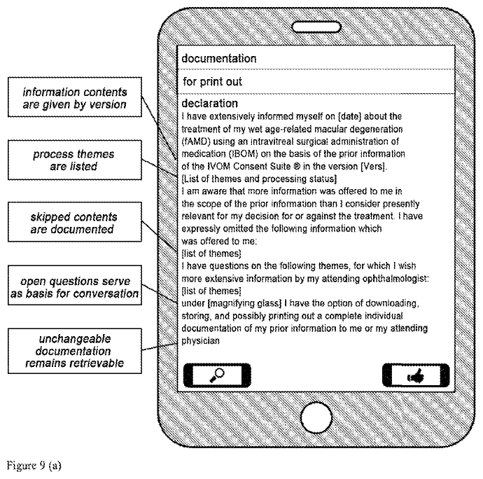

The functionalities described in the preceding sections are shown by way of example in FIG. 7. FIG. 7 shows schematically and by way of example a display on a computer system according to the invention, which is embodied as a smart phone or tablet computer. The user can perform various settings via a graphic user interface, which is displayed on a display screen of the computer system, which determine, inter alia, in which form information units are displayed.

All options which a user selects and all inputs which a user makes are preferably acquired and documented by the computer system according to the invention. Acquiring and documenting is understood to mean that the options and inputs of the user are registered and stored so that they can be reconstructed at a later point in time by the user and/or another/further person, so that it is apparent to the user and/or the other/further person which information units were consumed in which form and in which sequence, which questions, comments, and/or assessments were noted on individual information units, which information units were not output and/or were skipped, and/or how long the consumption of individual information units has lasted.

This acquisition and documentation serves, on the one hand, for the user so that he knows what he has processed, and when, and what is still open, etc.

On the other hand, the acquisition and documentation also serves for informing a further/other person who wishes to and/or has to gain an insight into which information units the user has consumed, and how. In the conveyance of information in the context of informed consent, this further/other person is generally a physician.

The physician can thus recognize which information units the user (patient) has skipped, in order to if necessary notify him once again of special risks or important circumstances which can be concealed in the skipped information. The physician can thus recognize in which information units the patient has had detailed indications displayed in order to possibly ask once again whether the patient has understood everything or has further questions. The physician can thus recognize which questions or comments the patient has input during the provision of information and can address them in a targeted manner. The physician can thus possibly recognize for which information units the patient has taken a comparatively long time (in comparison to other patients), to ask whether the patient has understood everything and/or requires further information. The physician can thus recognize if necessary which information units the patient has specifically assessed, for example, as too difficult or incomprehensible, to provide clarity here.

In other words, the acquisition and documentation of the user information, i.e., the information about the content and the manner in which the user has consumed the content, permit the physician in an individual information conversation:
to concentrate on the information units relevant for the patient,
to especially address the questions open for the patient,
to convey the information in a language and presentation preferred by the patient, and/or
to clarify the relevance of information units which the patient has expressly omitted.

The acquisition and documentation of the user information further also serves, however, to optimize the computer-assisted provision of information. For example, if there are information units which were skipped by a larger number of users than other information units, this can be an indication that the information unit is obsolete, redundant, or should be made more appealing. If there are information units which a majority of users have taken longer to consume than originally assumed/intended, it can be that these information units are too complicated and/or are particularly important for the user. It is conceivable that the information units should be reworked. If there are information units in respect of which a sufficient number of users have documented a need for additional information, it is conceivable to add additional more detailed information at the corresponding point.

Furthermore, the acquisition and documentation of the user information also serves for legal documentation of an information process in the context of an informed consent. If the patient has decided, for example, to skip an important information unit in spite of a notification of the importance, this is acquired and the patient cannot assert at a later point in time that important information was withheld from him.

FIG. 8 schematically shows by way of example information which have been acquired and documented during the conveyance of information by the computer system according to the invention and the computer program according to the invention. The information comprises the name of the user (Max Mustermann), the illness from which he suffers (age-related macular degeneration (AMD)), according to which information type the conveyance of information has taken place (standard information), the complexity of the language in which the information units have been conveyed (easy language), the estimated duration (12:04) for the consumption of the information units, the actual duration (14:09) for the consumption of the information units, the percentage proportion of the completed (consumed) information units, the number (2) of the questions which the user has noted for individual information units. Furthermore, themes and sections into which the information units are broken down are listed in the sequence in which they were consumed by the user. For individual themes (illness, progress of the disease of AMD, treatment of AMD), it is indicated how many information units (60 units) they comprise, and which percentage proportion thereof the user has consumed. It is indicated that the user has noted two questions on the theme of illness. The themes are further subdivided into sections (AMD, symptoms, new blood vessels, macular edema, natural course, treatment goals, treatment principle, active agents, treatment duration). It is also indicated for the individual sections what percentage the user has consumed thereof. The sections are furthermore subdivided into chapters. In the present representation, only the percentage consumed proportions in the form of a circle diagram are shown for the individual chapters. It is conceivable that further information about the chapters and further underlying breakdown levels can be retrieved, for example, by clicking on the circle diagrams by means of, for example, a mouse, a finger (on a touch-sensitive display screen), a pen, and/or the like. The acquired and documented information can be output to a physician so that the physician can complete the medical provision of information in a patient-specific manner.

In some countries, it is legally prescribed that the informed consent of a patient has to comprise an oral provision of information by the physician and/or a manual signature of the patient for the consent to carry out a medical measure and/or handing out documents (see, for example, the German Civil Code (BGB) § 630e).

Depending on the country in which the computer program according to the invention is used, functionalities and/or the scope of functionality of the computer program according to the invention can therefore be different.

In Germany, the computer program according to the invention is preferably intended to replace the first, paper-based level of the previous stepwise provision of information and prepare the second level in an individualized manner and/or assist its performance. The submission of the consent by the patient takes place thereafter in the conversation between physician and patient (second level).

The replacement of the previous paper-based first level of the stepwise provision of information by the solution according to the invention counters two of the still-existing points of criticism of the present patient information: individualized information is already given to the patient in the first level and the patient is relieved of burden, because the computer-assisted solution according to the invention no longer represents a total provision of information, rather the respective most important things are offered to the patient: “The provision of information has to take place in a patient-related manner and thus in accordance with the circumstances of the specific case and has to take into consideration recognizable decision preferences of the patient (see J. Lipinski: Vergleich der Aufklärung zur Intravitrealen Operativen Medikamenteneinbringung mit and ohne audiovisuelle Unterstützung [Comparison of the provision of information on intravitreal surgical administration of medication with and without audiovisual assistance], Inaugural dissertation to obtain a doctorate of the high medical faculty of the University of Cologne, 2016, section 1.1.1.).

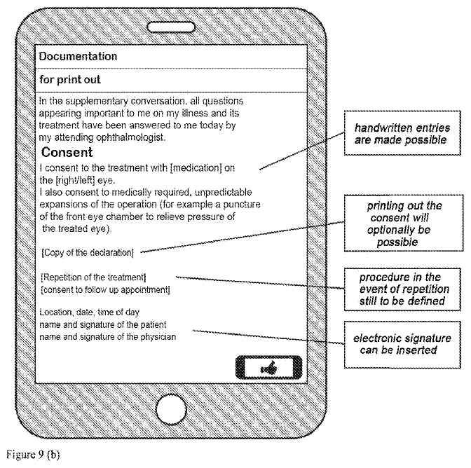

In one embodiment of the present invention, the consent and/or rejection of the upcoming medical treatment also takes place by means of the computer system/computer program according to the invention. The themes about which a patient has been instructed by means of the computer system/computer program according to the invention are preferably displayed to the patient in an overview. The information units which he has skipped are preferably displayed to him in an overview. The patient is preferably requested to confirm by actuating a virtual button or by a speech command that the provision of information has taken place as represented in one or more overviews. The upcoming medical treatment is preferably displayed to him in short form, and he can grant his consent to the medical treatment or reject it by actuating a virtual button or by a speech command. The functionalities described in this section are shown by way of example in FIG. 9(a) and FIG. 9(b). FIGS. 9(a) and(b) show schematically and by way of example displays on a display screen of a computer system according to the invention, which is embodied as a smart phone or tablet computer. Information is displayed to a user. The boxes on the left (in the case of FIG. 9(a)) or on the right (in the case of FIG. 9(b)) give further explanations about the displayed information.

It is also conceivable that the patient uses the computer program according to the invention in a first step himself (for example at home), to provide information to himself on an upcoming medical treatment. At the end of the computer-assisted provision of information, a file (for example a PDF file or another electronic text document) can be generated in which information about the provision of information is summarized (including the specification of the information units which the patient has consumed and which he has not consumed). The patient can, for example, print out the file on a printer (or have it printed out with the physician) and can personally sign it during or after the conversation with the physician. A scanned or photographed copy of the signed consent declaration can then be uploaded to the computer system (of the physician and/or the patient) for documentation and archiving.

In one preferred embodiment, the computer program according to the invention also comprises a functionality for filling out a questionnaire, for example, a medical history sheet. In this functionality, the patient is asked to answer questions on his person (age, sex, weight, size, blood type, and/or the like) and/or his medical prior history (for example previous illnesses) or to input corresponding information in the computer system according to the invention (e.g., via keyboard, mouse, touch-sensitive display, microphone, and/or the like). These can be output to the physician.

The computer system according to the invention can, as already described above, be designed, for example, as a tablet or desktop computer or smart phone. A terminal (kiosk), which is located in a doctor's office or a hospital, is also conceivable. The computer system according to the invention can be a single device, but it can also be a plurality of devices.

If it is a single device, it can be made available, for example, in the form of a tablet computer or a terminal to a patient, for example, in a doctor's office or a hospital. The patient can use the device to inform himself and in order to give a consent to the upcoming medical treatment if necessary.

However, a user can also load the computer program according to the invention on his computer system and use it there.

The computer system according to the invention can also comprise a plurality of devices, however, for example at least one for use by at least one patient and at least one for use by a physician. The computer system according to the invention preferably comprises at least two different devices, a first device and a second device, which are configured by two different computer programs to carry out the steps described hereinafter. This embodiment accordingly also comprises two different computer programs, a first computer program and a second computer program.

The first device is intended for use by a patient. There is preferably a plurality of first devices for a plurality of patients. The second device is intended for use by a physician. There is preferably a plurality of second devices for a plurality of physicians.

The first device is configured by the first computer program to output information to the patient in the form of information units. The patient can himself decide and control by corresponding selection and/or inputs in the first computer program which information units are output, which information units are not output (for example skipped), and in which form the information units are output.

The first device is furthermore configured to acquire which information units were output to the patient, which information units the patient has skipped and/or has not consumed, optionally how long he has taken for the consumption of individual information units, optionally which inputs (e.g., questions, comments, and/or assessments) the patient has made on individual information units, optionally in which sequence the patient has consumed information units, and/or the like (as described in this description at various points). The information acquired by the first device is also designated in this description as user information.

The first device is furthermore configured to transmit some or all user information to the second device, while the second device is configured by the second computer program to receive the user information transmitted from the first device. It is conceivable that the patient who operates the first device can decide in a defined scope whether and/or which user information is transmitted to the second device. The transmission of user information can thus take place, for example, automatically after consent of the user, or the contents to be transmitted can be selected by the user.

The transmission of user information from the first device to the second device can take place directly, for example, via a wired connection between the first device and the second device (for example, via a USB interface) or via a wireless connection between the first device and the second device (e.g., via a Bluetooth connection, via a WLAN connection, or via a mobile wireless connection).

The transmission of user information from the first device to the second device can also take place indirectly, however, for example, in that the first device stores user information in a cloud memory, while the second device reads the user information from the cloud memory.

The second device is furthermore configured to output the transmitted user information to the physician who operates the second device.

The first and/or the second device can be configured to accept a declaration of consent (for example for a medical treatment) in electronic form from the patient (for example in the form of an electronic or digital signature). It is conceivable that the physician also signs the patient consent; in such a case, the first and/or the second device can be configured to accept a signature from the physician in electronic and/or digital form.

The first and/or the second device can be configured to output a text document via a printer and to read in again the text document signed by the patient and optionally the physician. The first and/or the second device can be configured to store the signed text document, for example on a local memory and/or a cloud memory.

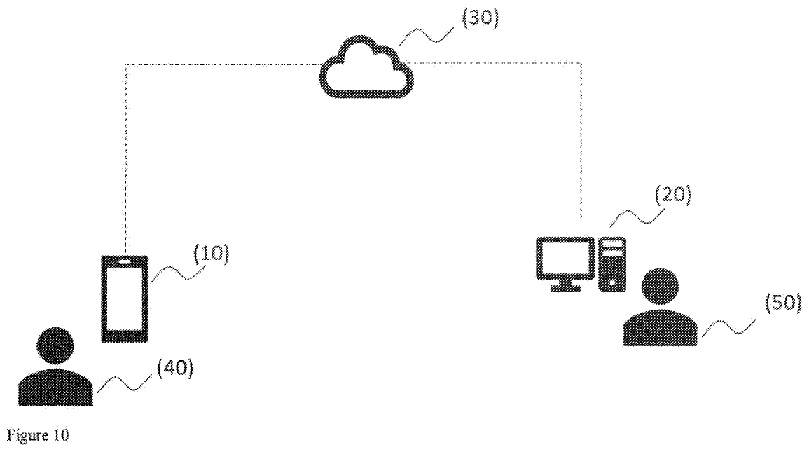

FIG. 10 schematically shows by way of example how at least one device (10), which is embodied as a smart phone or tablet computer, is operated by a patient (40). The patient (40) will be provided information by means of the computer program according to the invention, which is installed on the device (10) and is executed. Inputs of the patient (40) and further user information are acquired. If the patient has completed the provision of information and has optionally given (or declined) his consent to the medical treatment, the acquired information is transmitted via a network (shown here by the dashed line and the cloud server (30)) to a second device (20), which is operated by a physician (50) or another person, who works, for example, in a hospital or doctor's office. The physician (50) or the other person can check the user information, optionally check whether a consent for medical treatment is present, and/or prepare a conversation with the patient (40) on the basis of the transmitted information and/or initiate the medical treatment, for example, in that he sets an appointment for the conversation and/or the medical treatment and transmits it to the patient (40).

In a further preferred embodiment, the computer system according to the invention comprises, in addition to the at least one first and the at least one second device, at least one third device, which is used by a developer/operator of the computer program according to the invention. Via this at least one third device, the operator can provide the computer program for patients and/or physicians, perform maintenance, perform updates, and acquire anonymized information on the use of the computer program by physicians and patients in order to optimize the computer program—as described in this description.

Further embodiments of the present invention are:
1. A computer system for conveying medical information and for acquiring the type and the scope of the conveyed medical information, wherein the computer system is configured:
   to output information to a user in the form of information units,
   to receive inputs from the patient, wherein the inputs specify,
      which information units are to be output to the user,
      in which form the information units are to be output to the user, and
      whether the user has questions or comments on the information units,
   to output information units according to the inputs of the patient, wherein each information unit is designed such that it comprises written and/or spoken text, wherein the total number of the characters of the text in written form is less than 500,
   to acquire and document the inputs from the user, and
   to display the documented inputs to another/further person.
2. The computer system according to embodiment 1, wherein the information units are broken down into various themes and the inputs from the user furthermore specify how extensive information units will be on one theme or on multiple themes.
3. The computer system according to one of embodiments 1 or 2, wherein one or more time spans are output to the user, wherein the one or the more time spans indicate how long a consumption of one or more information units will require.
4. The computer system according to any one of embodiments 1 to 3, wherein the computer system is configured:
   to receive an input from the user, on one or on multiple information units, on skipping the one or the multiple information units,
   to give a notification to the user that the one or the multiple information units to be skipped are important information units,
   not to output the one or the multiple information units to be skipped if the user confirms the notification, and
   to acquire and to document the information units which are not output.
5. The computer system according to any one of embodiments 1 to 4, wherein the inputs on the form of the information units specify one or more of the following selections of the user:
   selection of a linguistic complexity of the information units,
   selection between output of the information units in the form of text and/or graphics and/or animations and/or images and/or video sequences, which are displayed on a display screen of the computer system, and/or output of the information units in the form of spoken language via a loudspeaker of the computer system,
   selection between input of commands and/or information into the computer system via a touch-sensitive display screen of the computer system and/or via a mouse of the computer system and/or via a keyboard of the computer system and/or via speech input via a microphone of the computer system,
   selection as to whether one or more knowledge tests are to be output to check the comprehension of the user,
   selection as to whether one or more avatars are to be incorporated.
6. The computer system according to any one of embodiments 1 to 5, wherein each information unit is designed so that its consumption occupies on average a time span of 5 seconds to 10 minutes, preferably 10 seconds to 5 minutes, still more preferably 10 seconds to 3 minutes.
7. The computer system according to any one of embodiments 1 to 6, wherein the computer system is configured to acquire the duration which the user requires to consume one or more of the output information units and to display it to the other/further person.
8. The computer system according to any one of embodiments 1 to 7, wherein the computer system is configured to send a message to the other/further person if the consumption of one or more information units has exceeded a defined duration.
9. The computer system according to any one of embodiments 1 to 8, comprising at least one first device, which is operated by the user, and at least one second device, which is operated by the other/further person, wherein the at least one first device is interconnected with the at least one second device via a network connection, wherein the at least one first device is configured to display the information units to the user and to receive inputs from the user and to transmit them to the at least one second device, wherein the at least one second device is configured to receive the information transmitted by the at least one first device and to output it to the other/further person.
10. The computer system according to any one of the embodiments 1 to 9, wherein the user is a patient, wherein the conveyance of information takes place in the context of an informed consent of the patient to an upcoming medical treatment, and wherein the other/further person is a physician.
11. A computer-implemented method for conveying medical information in the form of information units and for acquiring the type and the scope of the conveyed medical information, comprising the following steps:
   receiving inputs from a user, wherein the inputs specify,
      which information units are to be output to the user,
      in which form the information units are to be output to the user, and
      whether the user has questions and/or remarks on one or more information units,
   outputting the information units according to the inputs of the user, wherein each information unit is designed such that it comprises written and/or spoken text, wherein the total number of the characters of the text in written form is less than 500, and
   outputting the inputs of the patient to another/further person.
12. The method according to embodiment 11, furthermore comprising the following steps:
   receiving an input from the user, wherein the input specifies that one or more information units is/are not to be output to the user,
   outputting a notification that the one or more information units not to be output are one or more important information units,
   receiving an input from the user, wherein the input specifies that the user has been made aware of the notification and insists that the one or more important information units not be displayed,
   outputting the information units with the exception of the one or more information units for which the user has specified that it is/they are not to be displayed,
   acquiring the information units which were not output to the user, and
   outputting information to the other/further person of which information units were not output to the user.
13. The method according to any one of embodiments 11 to 12, furthermore comprising the following step:
   receiving one or more inputs from the user, wherein the one or more inputs determine how the information units are to be displayed, wherein the display comprises one or more of the following options:
      selection of a linguistic complexity,
      selection between output of the information units in the form of text and/or graphics and/or animations and/or images and/or video sequences, which are displayed on a display screen of the computer system, and/or output of the information units in the form of spoken language via a loudspeaker of the computer system,
      selection between input of commands and/or information via a touch-sensitive display screen and/or via a mouse and/or via a keyboard and/or via speech input via a microphone,
      selection as to whether one or more knowledge tests are to be output to check the comprehension of the user,
      selection whether one or more avatars are to be incorporated.
   outputting the information units to the user according to the selection of the user,
   outputting the selection of the user to the other/further person.
14. A computer program for conveying medical information in the form of information units and for acquiring the type and the scope of the conveyed medical information, comprising a computer-readable data carrier and program code, which is stored on the data carrier and which, upon execution on a computer system, causes the computer system to carry out the following steps:
   receiving inputs from a user, wherein the inputs specify,
      which information units are to be output to the user,
      in which form the information units are to be output to the user, and
      whether the user has questions and/or remarks on one or more information units,
   outputting the information units according to the inputs of the user, wherein each information unit is designed such that it comprises written and/or spoken text, wherein the total number of the characters of the text in written form is less than 500, and
   outputting the inputs of the user to the other/further person.
15. A method comprising using a computer system according to any one of embodiments 1 to 10 and/or a computer program according to embodiment 14 for conveying information and/or providing information to a patient in the context of an informed consent of the patient to an upcoming medical treatment, particularly preferably for the treatment of an age-related macular degeneration (AMD) by an intravitreal surgical administration of medication.

The present invention provides a valuable contribution to improving the informed consent of patients. As already shown by J. Lipinski in 2016 (see J. Lipinski: Vergleich der Aufklärung zur Intravitrealen Operativen Medikamenteneinbringung mit and ohne audiovisuelle Unterstützung[Comparison of the provision of information on intravitreal surgical administration of medication with and without audiovisual assistance], Inaugural dissertation to obtain a doctorate of the high medical faculty of the University of Cologne, 2016), “the use of an interactive audiovisual provision of information is possible even with older patients”. It “can result in added value for all participants in spite of, or in particular in the case of, impaired vision. Individualization of the patient information could thus begin with asking patients at the intake whether they would rather use a standardized paper information or an individualized software information. In contrast to the paper information, a digital solution can
guide the patient and require him to inform himself in the manner preferred by him and in accordance with his cognitive level about the decision to be made [ . . . ],
guide the patient and require him to select and weight typical and relevant preferences [ . . . ]
inform the patient well and prepare for the conversation so that a mutual decision-making is possible for the physician without additional time expenditure [ . . . ],
documenting the treatment goals of the patient and the treatment mandate of the physician in the office information system [ . . . ],
acquiring the information behavior, the understanding, the preferences, and the decision process of the patient in a legally robust manner [ . . . ],
securing the contents of the provision of information and the individual information process in the office information system in standardized form, and
inviting, guiding, or requiring the patient upon each new provision of information, on the basis of prior information and treatment experiences, to deepen his knowledge, consider his preferences, and take reintroduce them in the conversation with his physician [ . . . ].”

The computer program according to the invention is capable of documenting each step of the provision of information in detail, in particular also to which extent, which depth, and possibly with which success a patient has consumed the information units.

With the functionality of acquiring the decision process and consent of the patient, the computer program according to the invention can possibly also document in a legally binding manner that a patient “clearly and unambiguously states that he expressly dispenses with the information and he has accurately recognized the necessity for treatment and its prospects and risks” (see German parliament (2013), Law on improving the rights of patients. German Federal Law Gazette (9): 277-82) or he “has sufficient knowledge due to similar prior treatments, repeated equivalent measures, or his prior knowledge (http://dip21.bundestag.de/dip21/btd/17/104/1710488.pdf).”

1. A computer system for conveying medical information and for acquiring the type and the scope of the conveyed medical information in the context of an informed consent of a patient to a medical treatment, wherein the computer system comprises one or more processors configured to:
receive inputs from the patient, wherein the inputs specify:
information units to be output to the patient,
a sequence in which the information units are to be output to the patient, and
a form in which the information units are to be output to the patient;
output the information units according to the inputs of the patient, wherein each information unit comprises written and/or spoken text, wherein the total number of the characters of the text in written form is less than 500;
acquire information indicating:
which information units were or were not completely output to the patient and/or which information units the patient has skipped,
whether and possibly which information units were not output as a result of an input of the patient in spite of a notification of their importance,
whether and possibly which questions and/or comments and/or assessments the patient has given on one or more information units, and
how much time the patient has taken with consuming individual information units;
document the received inputs and acquired information; and
output the documented inputs and information to a physician.

2. The computer system of claim 1, wherein the information units are broken down into various themes and the inputs from the user comprise an indication of how extensive the information units will be on one theme or on multiple themes output to the patient.

3. The computer system of claim 1, wherein, for each information unit, the computer system is configured to determine how much time has passed between the beginning of the output of the information unit and the beginning of the output of a following information unit.

4. The computer system of claim 3, wherein the computer system is configured, from the determined time, to ascertain a consumption speed for the individual information units and/or to ascertain an arithmetically averaged consumption speed of the patient and to output it to the patient and/or the physician.

5. The computer system of claim 4, wherein the computer system is configured to compare the arithmetically averaged consumption speed for the individual information units to an average presentation speed and to output information to the physician indicating the extent by which the averaged consumption speed deviates from the average presentation speed.

6. The computer system of claim 1, wherein the computer system is configured, for the case that the consumption of one or more information units has exceeded a defined duration, to output information to the physician as to indicating the information units for which the consumption has exceeded the defined duration.

7. The computer system of claim 1, wherein the computer system is configured, for the case that the consumption of one or more information units has fallen below a defined duration, to output information to the physician as to indicating the information units for which the consumption has fallen below the defined duration.

8. The computer system of claim 1, wherein the computer system is configured to acquire the durations for which the individual information units have been output to the patient, to compare each of the durations to a reference time or multiple reference times, and, in the event of a defined deviation of an acquired duration from the one or the multiple reference times, to note, for the displayed information unit, that the patient has consumed or has not consumed the displayed information unit.

9. The computer system of claim 1, wherein the computer system is configured to:
receive, for one or more information units, an input from the user on skipping the one or more information units,
give a notification to the user indicating that the one or more information units to be skipped are important information units,
not output the one or more information units to be skipped if the user confirms the notification, and
acquire and to document the information units which are not output.

10. The computer system of claim 1, wherein the inputs on the form of the information units specify one or more of the following selections of the user:
selection of a linguistic complexity of the information units,
selection between output of the information units in the form of text and/or graphics and/or animations and/or images and/or video sequences, which are displayed on a display screen of the computer system, and/or output of the information units in the form of spoken language via a loudspeaker of the computer system,
selection between input of commands and/or information into the computer system via a touch-sensitive display screen of the computer system and/or via a mouse of the computer system and/or via a keyboard of the computer system and/or via speech input via a microphone of the computer system,
selection as to whether one or more knowledge tests are to be output to check the comprehension of the user,
selection as to whether one or more avatars are to be incorporated.

11. The computer system of claim 1, wherein each information unit is designed so that its consumption occupies on average a time span of 10 seconds to at most 3 minutes.

12. The computer system of claim 1, comprising a first device, which is operated by the user, and a second device, which is operated by a second person, wherein the first device is interconnected with the second device via a network connection, wherein the first device is configured to display the information units to the user, to receive inputs from the user and to transmit information associated with the received user inputs to the second device, wherein the second device is configured to receive the information transmitted by the first device and to output it to the second person.

13. The computer system of claim 1, wherein the computer system is configured to acquire movements and/or location changes of the computer system during the output of information units, to assign the acquired movements and/or location changes to a predefined profile, and to output information about the assigned profile to the physician.

14. A non-transitory computer readable storage medium storing instructions for conveying medical information and for acquiring the type and the scope of the conveyed medical information in the context of an informed consent of a patient to a medical treatment, which, upon execution by one or more processors of a computer system, causes the computer system to:
receiving inputs from the patient, wherein the inputs specify:
information units to be output to the patient,
a sequence in which the information units are to be output to the patient, and
a form in which the information units are to be output to the patient;
outputting the information units according to the inputs of the patient, wherein each information unit comprises written and/or spoken text, wherein the total number of the characters of the text in written form is less than 500;
acquiring the following information:
which information units were or were not completely output to the patient and/or which information units the patient has skipped,
whether and possibly which information units were not output as a result of an input of the patient in spite of a notification of their importance,
whether and possibly which questions and/or comments and/or assessments the patient has given on one or more information units, and
how much time the patient has taken with consuming individual information units;
documenting the received inputs and acquired information; and
outputting the documented inputs and information to a physician.

15. A computer-implemented method for conveying medical information and for acquiring the type and the scope of the conveyed medical information in the context of an informed consent of a patient to a medical treatment, the method comprising:
receiving, by one or more processors of the computer system, inputs from the patient, wherein the inputs specify:
information units to be output to the patient,
a sequence in which the information units are to be output to the patient, and
a form in which the information units are to be output to the patient;
outputting, by the one or more processors, the information units according to the inputs of the patient, wherein each information unit comprises written and/or spoken text, wherein the total number of the characters of the text in written form is less than 500;
acquiring, by the one or more processors, the following information:
which information units were or were not completely output to the patient and/or which information units the patient has skipped,
whether and possibly which information units were not output as a result of an input of the patient in spite of a notification of their importance,
whether and possibly which questions and/or comments and/or assessments the patient has given on one or more information units, and
how much time the patient has taken with consuming individual information units;
documenting, by the one or more processors, the received inputs and acquired information; and
outputting, by the one or more processors, the documented inputs and information to a physician.
